(19) United States
(12) Patent Application Publication
(10) Pub. No.: US 2008/0051764 A1
(43) Pub. Date: Feb. 28, 2008

(54) Physiological Monitoring With Continuous Treatment

(75) Inventors: Daniel L. DENT, San Antonio, TX (US); Jose Luis ALMEDA, Pasadena, CA (US)

Correspondence Address: MEYERTONS, HOOD, KIVLIN, KOWERT & GOETZEL, P.C., P.O. BOX 398, AUSTIN, TX, 78767-0398, US

(73) Assignee: BOARD OF REGENTS, THE UNIVERSITY OF TEXAS SYSTEM, Austin, TX (US)

(21) Appl. No.: 11/587,216

(22) PCT Filed: Apr. 19, 2005
(86) PCT No.: PCT/US05/13510

§ 371 Date: Sep. 24, 2007

Related U.S. Application Data
(60) Provisional application No. 60/563,350, filed on Apr. 19, 2004.

(51) Int. Cl. A61K 38/28

(52) U.S. Cl. 604/890.1; 514/3

(57) ABSTRACT

The system and method may measure, monitor, and control physiological conditions in a body. In some embodiments, the system may include a flexible conduit (100), one or more sensors (181), and a processor. A flexible conduit may be positioned in the vasculature or a cavity of a body. Sensors may measure one or more physiological conditions and a processor may generate one or more responses in reaction to measured physiological conditions. In certain embodiments, responses may include delivery of pharmaceuticals and/or altering machine functions.

### BACKGROUND

1. Field of Invention

The present invention relates to systems and methods for measuring, monitoring, and treating physiological changes in a human body. More particularly, the invention relates to systems and methods for automatically monitoring physiological conditions intravascularly or within body cavities and automatically treating changes in physiological conditions.

2. Description of Related Art

Tight control of human blood glucose near physiologic normal may help decrease morbidity and mortality for Intensive Care Unit (ICU) patients. Normal blood glucose levels may contribute to a decrease in length of stay and aid wound healing. For both diabetics and non-diabetics with hyperglycemia in a critical care setting, maintaining normal blood glucose levels usually involves a nurse titrated intravenous drip of regular insulin based largely on nonscientific protocols.

Glucose levels may be measured via interstitial compartments or subcutaneous capillary finger pricks. Some ICU patients have substantial peripheral edema rendering interstitial or subcutaneous sensors less than ideal. Some subcutaneously implanted sensors, such as a permanent artificial pancreas, have been used in human and animal models that monitor interstitial fluid, assuming a correlation between glucose levels in blood and that of the intravascular space. Sensors in these applications have to be frequently recalibrated, surgically implanted, and later surgically removed based on product duration. The power source must be internal and the entire device must also be biocompatible. In some situations, however, critically ill diabetics and non-diabetics may need acute glucose management that takes into account intravascular and extravascular fluid changes and fluctuating electrochemical gradients.

In effect, however, blood glucose levels are commonly measured using standard laboratory analysis or bedside devices designed for home insulin management. Based on hourly (or less frequent) results, an insulin rate may be titrated based on a set protocol followed by nursing staff. Due to the stress of illness and/or trauma, blood glucose levels may be in constant wide range fluctuation, and only modest control of blood glucose levels may be achieved in this manner. In addition, obtaining hourly glucose measurements and manually adjusting the rate of insulin infusion is extremely labor intensive for the nursing staff.

Furthermore, maintaining other physiological conditions near physiologic normal may help decrease morbidity and mortality for Intensive Care Unit (ICU) patients, surgical patients, medical patients, and burn patients. While many physiological conditions may be monitored periodically in a hospital setting, doctors and nurses are required to read measured physiological conditions and determine the appropriate response.

### SUMMARY

In an embodiment, a regulatory system may monitor physiological conditions in a human body. A regulatory system may maintain one or more physiological conditions in a desired range. A desired range may be determined by FDA guidelines. A regulatory system may include a flexible conduit, one or more sensors, and a processor. A flexible conduit may include an intravenous catheter and/or a central line. A portion of flexible conduit may be positionable in a body. A portion of flexible conduit may be positionable in the vasculature or a cavity of a human body. One or more sensors may be positionable on a flexible conduit. In an embodiment, a sensor may measure one or more physiological conditions in a body. A processor coupled to sensors may generate one or more responses to a measured physiological condition.

In one embodiment, a response may include controlling one or more machines and/or adjusting one or more machine functions. Machines adjusted by a processor may include a ventilator and/or a pacemaker. A response may include delivering one or more pharmaceuticals to a body. Pharmaceuticals may be stored in a pharmaceutical storage unit. A response may include delivering pharmaceuticals from a pharmaceutical storage unit. In an embodiment, a pharmaceutical selected may alter, modify, and/or adjust at least one physiological condition. A pharmaceutical may be delivered such that a physiological condition is modified to be in a desired range.

In one embodiment, blood glucose levels may be controlled by a regulatory system. Insulin may be delivered as a response to high measured blood glucose levels.

A regulatory system may control blood pressure in a body. A response to high or low measured blood pressure levels may include delivery of blood pressure medication. A pharmaceutical may be configured to modify blood pressure to within a desired range.

A regulatory system may measure one or more physiological conditions in a body using one or more sensors. Values for measured physiological conditions may be compared to a desired range or normal values for a physiological condition. A processor may automatically control measured physiological conditions to be substantially in a desired range or range of normal values for a body. In an embodiment, a regulatory system may monitor and control a plurality of physiological conditions. A display on a processor may exhibit values for measured physiological conditions.

### BRIEF DESCRIPTION OF THE DRAWINGS

Advantages of the present invention will become apparent to those skilled in the art with the benefit of the following detailed description of embodiment and upon reference to the accompanying drawings, in which:

FIG. 1 depicts an embodiment of a regulatory system.

FIG. 1a depicts an embodiment of a flexible conduit positioned in an artery.

FIG. 2 depicts a flow chart of a method of measuring, monitoring, and controlling physiological conditions with an embodiment of a regulatory system.

While the invention is susceptible to various modifications and alternative forms, specific embodiments thereof are shown by way of example in the drawings and will herein be described in detail. It should be understood that the drawing and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION

Herein we describe a system and method for measuring, monitoring, and treating physiological changes in a human body. Physiological conditions, in the context of this application, refer to characteristics of the functioning of a human body. Physiological conditions include, but are not limited to, glucose levels; insulin levels; blood pressure; ion concentrations (such as potassium levels, magnesium levels, phosphorous levels, and electrolyte concentrations); carbon dioxide levels; blood oxygen levels; pharmaceutical levels (such as chemotherapy drug levels and/or antibiotic levels in a body); intracranial pressure; measures of blood clotting; and pH.

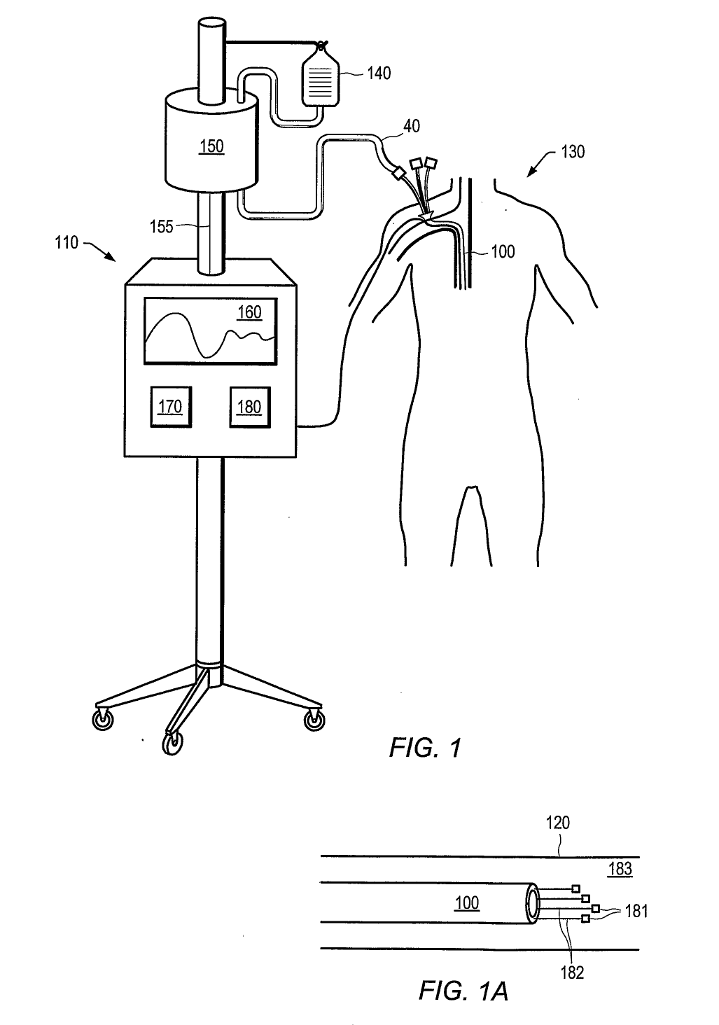

The regulatory system and method measure physiological conditions at a site in a human body. A regulatory system may be optimized to maintain homeostasis and/or alter physiological conditions in a body to substantially achieve homeostasis. In some embodiments, a regulatory system may include a flexible conduit 100 with one or more sensors and a processor 110, as shown in FIG. 1. Flexible conduit 100 may be positionable in a vasculature 120 of a human body 130 (as depicted in FIG. 1A). Sensors may measure a physiological condition in a body. Sensors may be coupled to a processor 110. In certain embodiments, a processor may monitor measured physiological conditions. A processor may generate one or more responses. Responses may result in the alteration and/or modification of at least one measured physiological condition. A response may include a delivery of pharmaceuticals from a pharmaceutical storage unit 140 and/or altering a machine function. A pump 150 may facilitate delivery of pharmaceuticals from a pharmaceutical storage unit 140. Processor 110 may be coupled 155 to pump 150. A processor 110 may alter the rate at which a pump 150 operates in response to a measured physiological condition. A processor 110 may display a graph 160 and/or values for measured physiological conditions 170, changes in measured physiological condition, and/or rates of pharmaceutical delivery 180.

A flexible conduit may be made of a physiologically inert material. Flexible conduit may be made of latex, silicone, plastic, metal, and/or combinations thereof. Flexible conduit may be configured such that, during insertion in a body, a lumen of the flexible conduit may not substantially collapse. In an embodiment, a portion of the flexible conduit may be rigid. Flexible conduit may be configured to substantially withstand temperature, pressure, and/or pH changes in a body. A flexible conduit may be at least partially made of one or more radio-opaque materials. A flexible conduit may include radio-opaque markers that facilitate verification of placement of the flexible conduit.

A flexible conduit may have a shape similar to catheters known to one skilled in the art. In some embodiments, a flexible conduit may have a substantially circular, substantially oval, substantially rectangular, or irregular cross sectional shape. In an embodiment, a cross-section of a flexible conduit may change along a length of the flexible conduit. Flexible conduit may be a tube. Flexible conduit may be a single, double, triple, or other multiple lumen catheter. In one embodiment, flexible conduit may be a 7.5 French triple lumen catheter. Flexible conduit may be an intra-arterial catheter. Flexible conduit may be an intra-cranial pressure monitoring device.

In an embodiment, a flexible conduit may be positionable in the vasculature or a cavity of a human body. A vasculature of a human body, in the context of this application, refers to the network of blood vessels in a human body. A flexible conduit may be positionable in an artery. A flexible conduit may be positionable in a femoral vein. Flexible conduit may be an intravenous catheter, a pulmonary catheter, a central venous catheter, or a peripheral venous catheter. Flexible conduit may be a Groshong® catheter, commercially available from Bard Access Systems. Flexible conduit may be an Intrasil catheter, commercially available from Baxter Healthcare Corporation. Flexible conduit may be a Swan Ganz catheter. Flexible conduit may be positioned in the urinary bladder such as a Foley or other urinary catheter. Flexible conduit may be positioned in the cranium such as in intra-cranial pressure monitor. Flexible conduit may be positioned in an artery and may include an Arrow arterial line catheter.

In certain embodiments, a flexible conduit may include a filter. A filter may substantially inhibit blood clot formation proximate and/or in a flexible conduit. A flexible conduit may include a valve configured to substantially inhibit blood clot formation proximate and/or in a flexible conduit. In an embodiment, a blood thinner, such as heparin, may be injected in and/or otherwise delivered to a flexible conduit to substantially inhibit blood clot formation proximate a flexible conduit. In one embodiment, a flexible conduit may be flushed with saline. Flushing a flexible conduit with saline may inhibit microorganism growth, substantially prevent accumulation of pharmaceuticals in a lumen of the flexible conduit, and/or inhibit blood clot formation.

In some embodiments, one or more sensors may be positionable in a flexible conduit. In certain embodiments, a plurality of sensors is positioned in a flexible conduit. Alternatively, one or more sensors may be coupled to a flexible conduit. Coupling sensors to a flexible conduit, in the context of this application, refers to fastening two or more items together, linking two or more items together, and/or bringing two or more items in such close proximity as to permit mutual influence. Sensors may be bonded, glued, and/or otherwise affixed to a flexible conduit. Sensors may be implanted on a flexible conduit. In an embodiment, sensors may not be coupled to a flexible conduit. Sensors may be positionable in an opening in a flexible conduit. In an embodiment, sensors may be positioned proximate an end of a flexible conduit. In one embodiment, at least a portion of a sensor may be positionable outside flexible conduit.

In some embodiments, one or more sensors may be coupled proximate an end of a probe. A probe may be a wire, tube, and/or rod with any cross-sectional shape. Probes may have a diameter smaller than a diameter of a flexible conduit. A probe may be positionable in a flexible conduit. A probe may be partially positioned in a flexible conduit. A plurality of sensors may be positionable on a probe. In certain embodiments, a plurality of probes may be positionable in a flexible conduit. In one embodiment, one or more probes may be coupled to flexible conduit. Probes may be placed in a flexible conduit and then coupled to the flexible conduit. A user may place a flexible conduit at a desired location in a vasculature or in a body cavity and then position one or more probes in the flexible conduit. Probes included in a flexible conduit may be patient dependent. In an embodiment, more than 5 probes may be positioned in a flexible conduit. More than 50 probes may be positionable in a flexible conduit.

As depicted in FIG. 1a, a flexible conduit 100 may be positioned in an artery 120 in a body. Sensors 181 may be coupled to probes 182. Probes 182 may be at least partially positioned in a flexible conduit 100. Sensors 181 coupled to an end of a probe 182 may be positioned such that sensors 181 contact a bloodstream 183 in the artery 120.

Sensors may measure one or more physiological conditions in a body. Monitoring physiological conditions in vivo may have advantages over other monitoring techniques including, but not limited to, more accurate measurements of levels of ions, pharmaceuticals, and/or blood pressures. A body may be subject to less trauma by in vivo monitoring since blood may not have to be repeatedly drawn from a body. In some embodiments, measurements that may not be obtainable external to the body may be obtained by in vivo measurement including hemodynamic properties and/or ion concentrations in certain regions of a body.

One or more sensors may be fiber optic based sensors. In another embodiment, one or more sensors may use near infrared evanescent spectrophotometry. Sensors may measure ion concentration, blood pressure, intracranial pressure, glucose concentration, and/or pH. Sensors may be biosensors. Sensors may include nanotechnology. Sensors may also be used for hemodynamic monitoring and monitoring pharmaceutical levels in body.

A sensor may be an optical glucose sensor as described in U.S. Pat. No. 6,627,177 to Singaram et al and U.S. Patent Application Publication No. 2004-0028612 to Singaram et al. An optical glucose sensor may glow in the presence of glucose and/or detect a level of glucose in a human body. A sensor may be a sensor for a filmless radiography system as described in U.S. Pat. No. 6,652,141 to Cianciosi.

In certain embodiments, a regulatory system may monitor glucose levels in a body. Sensors in the regulatory system may continuously measure glucose levels. Sensors may be enzymatic biosensors. Enzymatic biosensors may include immobilized glucose oxidase as the enzyme that catalyzes oxidation of glucose to gluconic acid with the formation of hydrogen peroxide. Enzymatic biosensors may measure glucose levels by oxygen consumption, hydrogen peroxide production, and/or electron transfer.

In some embodiments, a sensor that measures glucose levels by oxygen consumption may include a cylindrical sensor in which glucose and oxygen flow to a sensor and an enzyme. Glucose and oxygen may diffuse into the enzyme region of the sensor from one direction, but only oxygen may be allowed to diffuse from the other direction. The oxygen consumed by the enzyme may be correlated to glucose levels in a body.

In certain embodiments, hydrogen peroxide production may be used to measure glucose levels. Sensors capable of measuring hydrogen peroxide may measure the product of an enzymatic reaction on an anodically polarized electrode. As glucose concentrations increase, the signal from the electrode may increase. Semipermeable membranes proximate the electrodes may reduce interference by restricting passage of species that are electrooxidizable by the applied potential.

In one embodiment, sensors may use species other than oxygen to transfer electrons to the enzymatic electrode. A body may have a low ratio of oxygen to glucose. Circumventing the oxygen deficit in a body may be possible by using a species other than oxygen to transfer the electrons from glucose oxidase to an electrode. Glucose oxidase may be coupled to an electrode with a hydrogel including a redox polymer with an electrochemically active and a chemically bound complexed osmium redox center.

In some embodiments, a glucose sensor may be a fiber optic probe that uses near-infrared evanescent spectrophotometry (NIRS). A fiber optic probe may be positionable in a flexible conduit. NIRS may include illumination of a vascular tissue component with a broad-band energy emitted by a source through a fiber-optic cable. Energy may be scattered, absorbed, and/or transmitted in several directions in a measured volume and collected by a detection fiber optic cable. NIR spectra from a fiber optic cable may represent molecular vibrations associated with more than one biological constituent. NIR spectra obtained by sensors may be processed using multivariate algorithms. An algorithm may create a calibration model that relates spectral information obtained by the fiber optic probe to a concentration of glucose in the blood.

Sensors may be coupled to a processor. Coupling sensors to a processor, in the context of this application, refers to directly attaching the sensors to the processor, coupling the sensors to the processor through other members of the regulatory system, and/or coupling the sensors to the processor through wireless electronic transfer. Sensors may be coupled to wires, which transmit measured physiological conditions to a processor. In some embodiments, wireless electronic transfer may transmit measured physiological conditions to a processor.

In certain embodiments, other sensors external to a body may be coupled to a processor. Other sensors may be positioned on a surface of the body. Wires may couple the other sensors to the processor. Other sensors external to the body may include EKG leads, anesthesia twitch monitors, EEG leads, pulse oximeters, and/or other sensors commonly used to monitor physiological conditions external to a body. Other sensors may include non-invasive sensors capable of monitoring physiological conditions. A processor may use information from the sensors in one or more flexible conduits and/or other sensors external to a body to monitor physiological conditions, generate responses, and/or display data received from the sensors.

In some embodiments, a processor may receive and/or display data on measured physiological conditions from sensors. A processor may receive data from wires coupled to sensors and/or from wireless transfer of data. A display (e.g., display 160 depicted on FIG. 1) on a processor may include a CRT display, a LCD display, a plasma display, a moving or stationary strip of paper, a document, light emitting diodes, and/or other devices capable of exhibiting measured physiological conditions and/or changes thereof. A display may show one or more responses generated by a processor. A display may continuously show measured physiological conditions, changes in physiological conditions, and/or a range a measured physiological condition is in (e.g., normal, above normal, critical, below normal, etc.). When a measured physiological condition deviates from a range, a display may show the deviation from the range and/or an alarm may signal. An alarm may include, but is not limited to, a warning light, a sound, or a message transmitted by wireless electronic transfer. A display may include a graph of values for continuously measured physiological conditions. In an embodiment, a display may graph glucose levels and insulin delivery rates over time. In other embodiments, a display may graph a physiologic parameter being measured and pharmaceutical or intervention being delivered over time.

A processor may monitor changes in measured physiological conditions. A processor may generate one or more responses at least partially in reaction to a measured physiological condition. In some embodiments, a processor may include an algorithm or software program to facilitate determination of the appropriate response. An algorithm may be designed to produce responses that promote homeostasis. Algorithm may be facilitated to control physiological conditions at a predetermined level in a closed loop system. Algorithm may account for patient specific factors and illness specific factors that may influence selection of an appropriate response.

In some embodiments, a processor may compare a measured physiological condition to a desired range associated with the measured physiological condition. In an embodiment, a desired range may be determined by FDA guidelines. A desired range may be values for the measured physiological condition previously obtained (e.g., prior to infusion of a pharmaceutical or prior to surgery). A desired range may be a range of values for the measured physiological condition in normal humans. A desired range may be inputted into a processor by an operator. In an embodiment, desired ranges for a plurality of physiological conditions may be uploaded to a processor concurrently. In one embodiment, desired ranges for a plurality of physiological conditions may vary with time. A processor may intermittently or continuously upload updated desired ranges for a body. In one embodiment, maintaining a physiological condition in a desired range comprises maintaining a physiological condition within a small tolerance of an optimum level for the physiological condition.

In certain embodiments, a processor generates one or more responses in reaction to a measured physiological condition. A processor may generate one or more responses when a measured physiological condition does not substantially deviate from a previous value for the measured physiological condition. A processor may generate one or more responses when a measured physiological condition deviates by a pre-determined range. In one embodiment, a processor may generate one or more responses when a measured physiological condition deviates from a desired range for the measured physiological condition. In an embodiment, a processor may not generate a response to a measured physiological condition. A processor may automatically control at least one measured physiological condition so that the measured physiological condition is maintained substantially in a desired range.

A response may include initiating further monitoring. In an embodiment, further monitoring may include measuring physiological conditions in which continuous monitoring may not be desirable due to, for example, toxicity, cost of testing, and/or cost of pharmaceuticals involved in testing.

One or more responses may include delivery of one or more pharmaceuticals to a human body. A response, in the context of this application, is a reaction to a measured physiological condition. A response may alter and/or modify a measured physiological condition. In one embodiment, a response may include continuing pharmaceutical delivery at a rate similar to a rate prior to measurement. A response may include not substantially changing pharmaceutical delivery rates or machine functions. Pharmaceuticals, in the context of this application, are any compound or combination of compounds that produce a physiological change when administered to a body. A pharmaceutical may include, but is not limited to, a therapeutic agent.

Pharmaceuticals may be delivered by a FDA approved device. Pharmaceuticals may be delivered by a standard FDA approved intravenous delivery device. In some embodiments, a flexible conduit of a regulatory system may deliver a pharmaceutical. Pharmaceuticals may be delivered via an outer lumen of a flexible conduit and one or more sensors may be positioned in an inner lumen of a flexible conduit or vice versa. Flexible conduit may include two or more separate lumens. In an embodiment, pharmaceuticals may be delivered in a first lumen of a flexible conduit, while sensors may be positioned in a different lumen. In one embodiment, a regulatory system includes a triple lumen catheter in which pharmaceuticals are delivered through one lumen and sensors are positioned in one or both of the other lumens. Alternatively, pharmaceuticals may be delivered and sensors may be positionable in the same lumen. In an embodiment, pharmaceuticals may be delivered via a pharmaceutical delivery conduit coupled to a human body. A pharmaceutical delivery conduit for pharmaceutical delivery may be subcutaneous or intravascular.

In some embodiments, generating a response by a processor may include delivery of one or more pharmaceuticals to a human body. Pharmaceuticals may be housed in a pharmaceutical storage unit (e.g., storage unit 140, depicted in FIG. 1). A pharmaceutical storage unit may include several components housed separately. A processor may be coupled to a pharmaceutical storage unit. In some embodiments, a processor may be in a pharmaceutical storage unit. One or more pumps may be coupled to a pharmaceutical storage unit to delivery pharmaceuticals from a pharmaceutical unit to a body. A pharmaceutical storage unit may include one or more chambers. A pharmaceutical may be stored in one or more chambers. In one embodiment, a pharmaceutical storage unit may include several subunits comprising chambers capable of independently storing and/or delivering pharmaceuticals.

A pharmaceutical storage unit may be capable of holding a plurality of pharmaceuticals. A pharmaceutical storage unit may be configured to substantially inhibit different pharmaceuticals from leaking or mixing with other pharmaceuticals. A pharmaceutical storage unit may be refillable. In an embodiment, chambers may be replaceable. Pharmaceuticals may be sold in prefilled chambers and the chambers may be replaced when an amount of pharmaceutical in a chamber is less than desired. In an embodiment, to fill a chamber with a pharmaceutical, a container at least partially filled with a pharmaceutical may be inserted into a chamber.

Generating one or more responses may include modifying and/or altering a machine function. In some embodiments, machines, such as ventilators, pacemakers and pumps, may be controllable by a processor. When a measured physiological condition changes, a processor may change a machine function, where the machine is configured to modify and/or alter the measured physiological condition. In certain embodiments, responses generated by a processor may include altering a machine function and delivering a pharmaceutical to a body. A response may be altering a machine function, such as a ventilator rate, a tidal volume, a pressure setting, or an amount of oxygen delivered from a ventilator.

In certain embodiments, when glucose levels in a body rise above a desired range, a processor may generate a response including delivering insulin from a second chamber of a pharmaceutical storage unit. A pharmaceutical storage unit may include an insulin pump to deliver insulin at a desired rate. Potassium may similarly be delivered. When an amount of pharmaceutical in a pharmaceutical storage unit chamber is below a minimum level, a pharmaceutical storage unit may indicate the low pharmaceutical level. A pharmaceutical storage unit may indicate low pharmaceutical levels with one or more lights, one or more light emitting diodes, one or more sounds, one or more warnings on a display, or by indicating to a processor that the pharmaceutical level is low. A processor may then indicate the low pharmaceutical level with an alarm including: sounds, lights, light emitting diodes, and/or one or more message sent to a user.

A processor coupled to sensors may continuously monitor physiological conditions. In some embodiments, a regulatory system may maintain a measured physiological condition in a desired range. In certain embodiments, when a physiological condition deviates from a desired range, a processor generates one or more responses. At least one response is configured to modify the measured physiological condition. A response may include delivering pharmaceuticals to a body. In an embodiment, pharmaceuticals may be continuously infused into a body. A processor may calculate subsequent doses of pharmaceuticals to be delivered based on previously measured physiological condition values and previous pharmaceutical delivery rates. A processor may calculate the next dose of pharmaceuticals or the appropriate response continuously and in real time. In certain embodiments, a regulatory system may be a closed-loop control system where one or more physiological conditions are maintained substantially in a desired range and where a processor automatically adjusts responses (e.g., pharmaceutical delivery rates, machine functions, etc.).

A processor may analyze characteristics of a measured physiological condition to calculate an optimum next dose. In some embodiments, a measured physiological condition, x, may be plotted versus time, t. A response generated by a processor may be a function of the variation of x over time (e.g., the derivative of the measured physiological condition curve). A processor may analyze the variation of x over time (e.g., the derivative of the measured physiological condition curve, f(x)) to generate the appropriate response. In certain embodiments, a response, r, such as pharmaceutical delivery amounts may be plotted versus time, t. The integral of the response curve, f(r), between a time, tₒ and any time, t_{z}, may represent a total amount of pharmaceuticals delivered to a body during the time interval of tₒ to t_{z}. A processor may continuously calculate changes such as, slopes of curves and deviations in responses generated over time to predict the optimum next dose. Optimum pharmaceutical delivery rates may be a function of factors including, but not limited to, total pharmaceutical delivered over time, change in machine function over time, response of a body to last dose of pharmaceutical, change in measured physiological condition over time, and/or pharmaceutical diffusion rates in blood, tissue, or musculature of body.

A flexible conduit may be positioned in a small peripheral vein or a larger more central vein including, but not limited to, the external jugular vein, the internal jugular vein, the subclavian vein, the inferior vena cava, and/or the superior vena cava. A flexible conduit may be a central venous catheter inserted in an operating room. In some embodiments, a flexible conduit is inserted proximate an elbow and into a vein. A flexible conduit may be inserted proxinate a shoulder and into a vein. In some embodiments, a user may insert a needle through the skin and into a vein in a body. A guide wire may pass through the needle and be used to direct a flexible conduit to a desired location in a vasculature of a body. A guide wire may be removed after positioning flexible conduit in a desired location. In certain embodiments, an incision may be created in the skin and a flexible conduit may be inserted and placed in a desired location in a vein. A flexible conduit may be placed in a body cavity including the head, the abdomen, or the thorax. A flexible conduit may be at least partially positioned in a specific organ including the stomach, the urinary bladder or the small intestine. A flexible conduit may be secured in a desired location. In an embodiment, a portion of the flexible conduit may be outside a body. A portion of a flexible conduit may pass through a small section of the skin prior to positioning the flexible conduit in a vein in a body. An x-ray may be used to verify proper positioning of a flexible conduit. In an embodiment, a blood thinner may be injected into a flexible conduit to prevent blood clot formation proximate flexible conduit.

In some embodiments, a plurality of sensors may be positioned in a flexible conduit. Flexible conduit may be positioned in the vasculature of a body. Sensors may be directly or indirectly (e.g., via wireless electronic transfer, via other components of the system, etc.) coupled to a processor. In certain embodiments, multiple flexible conduits are positioned in a body. A processor may be directly or indirectly coupled to sensors positioned in one or more flexible conduits and/or other sensors not positioned in flexible conduits. In an embodiment, other sensors may monitor physiological conditions subcutaneously or externally. A processor may generate one or responses in reaction to measured physiological conditions of sensors not positioned in a flexible conduit and/or the plurality of sensors in one or more flexible conduits.

In some embodiments, pharmaceutical storage unit may include at least a first, second, and third chamber. A first chamber may be at least partially filled with glucose. A second chamber may be at least partially filled with insulin. A third chamber may be at least partially filled with potassium. A plurality of sensors in a flexible conduit may, at least, measure glucose and potassium levels in a body. In certain embodiments, sensors may be coupled to probes positioned in a flexible conduit.

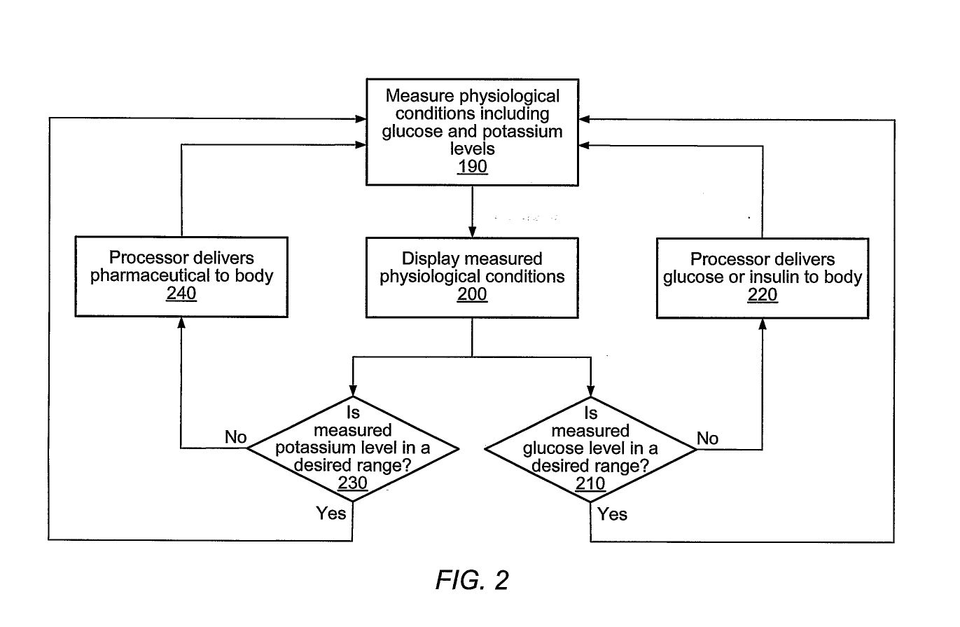

During use, sensors may continuously measure a plurality of physiological conditions including glucose and potassium levels 190, as shown in FIG. 2. Sensors may measure true serum glucose levels. A processor coupled either directly or indirectly to sensors may monitor physiological conditions detected by sensors, including glucose and potassium levels. A processor may display measured physiological conditions 200. Processor may compare measured glucose levels to a desired range 210. When glucose levels in the body are below a desired range, a processor may generate a response including delivering glucose and/or insulin from a pharmaceutical storage unit to a body 220. A processor may utilize an algorithm to determine an appropriate rate of glucose and/or insulin delivery. An algorithm may be a function of total glucose delivered to a body, diffusion rate of insulin and/or glucose in a vasculature, present insulin delivery rate, and/or values of other physiological conditions. Glucose may be delivered to a body continuously for a pre-determined time period or intermittently. Additionally, processor may compare measured potassium levels to a desired range for potassium levels in a body 230. If potassium levels are outside the desired range, a pharmaceutical may be delivered to a body 240. In an embodiment, a processor may monitor potassium infusion rates and total amount of potassium infused. Closely monitoring glucose and potassium levels may be beneficial to patients with diabetic ketoacidosis, which may be encountered in an ICU setting. Changes in the rates of glucose, insulin, and potassium delivered may be based on continuous averages of serum potassium and glucose changes. Changes in rates of glucose, insulin, and potassium delivered may be based on point in time measurements. Sensors may continue to monitor physiological conditions during and after pharmaceutical delivery 190.

A regulatory system may be used to monitor blood glucose levels in a body. An intravascular probe may detect true serum glucose levels without having to draw blood from a patient. In an embodiment, a regulatory system includes one or more enzymatic glucose sensors. One or more sensors configured to measure glucose may be biosensors. One or more sensors configured to measure glucose may include near IR spectrophotometry. Sensors configured to measure glucose may include impedance spectroscopy. Glucose variations and/or trends may be closely analyzed to calculate a dose of insulin based on more than one blood glucose measurement. In an embodiment, the next dose of insulin to be delivered to a body may be a function of more than one glucose measurement. A plurality of glucose measurements may be used to determine glucose trends and glucose variations. The dose of insulin delivered may be related to the glucose trends and variations. By maintaining continuous intravascular monitoring with real time continuous adjustment of insulin infusion, nursing workload may be reduced, patients may be maintained in better physiological homeostasis, patient length of stay may decrease, wound healing may increase, and/or morbidity and mortality for patients overall may decrease, when compared to manual insulin titration. In certain embodiments, a regulatory system may be a closed-loop control system where blood glucose levels are maintained substantially in a desired range and where a processor automatically adjusts insulin delivery rates. A processor may continuously calculate changes in glucose levels in blood over time and/or deviations in infused insulin rates over time to predict the optimum next dose.

A regulatory system may be used to maintain a desired glucose level in a body. In one embodiment, glucose levels may be monitored and controlled at a level less than glucose levels in a normal human. Regulatory system may be used to determine an optimum level of glucose in a body for critically ill patients. In critically ill patients, glucose may be maintained at a level lower than a normal human glucose level. Glucose levels may be maintained below normal human glucose levels by infusing large amounts of serum insulin. In an embodiment, maintaining a low glucose level may have a protective function on a body and/or decrease damage by decreased oxygen delivery and/or sepsis.

In some embodiments, a regulatory system may be used to monitor levels of pharmaceuticals in a body. In some embodiments, it may be desirable to control the level of pharmaceuticals in a body in a desired treatment range. One or more sensors may measure concentrations of pharmaceuticals in a body. The treatment range may be an optimum level of pharmaceuticals in a body. The treatment range may be configured to comply with FDA guidelines. Pharmaceuticals may include, but are not limited to, phenytoin, benzodiapepines and other sedatives, Propofol, narcotics, antibiotics, chemotherapeutic agents, amino acids, inotropes, cardiovascular agents, anticoagulants, and/or inflammatory mediators or blockers.

A regulatory system may monitor and control levels of an antibiotic in a body. An antibiotic may be continuously infused. By continuously monitoring antibiotic levels, an optimum level of antibiotics in blood for various infections may be determined. Continuous monitoring of antibiotic levels may also determine the optimum level of antibiotics in a body with low occurrence of side effects. In certain embodiments, antibiotics used for prolonged periods of time at high doses may become toxic, such as aminoglycosides and vancomycin. A processor of a regulatory system may intermittently drop levels of antibiotics delivered to a body such that toxic effects are minimized. A regulatory system may be similarly used in chemotherapy agent applications.

In an embodiment, urinary output may be monitored by a regulatory system. A regulatory system may include one or more flexible conduits including a Foley catheter. Sensors in the Foley catheter may monitor urinary output. Urinary output may be controlled in a desired range by a processor. A processor of a regulatory system may generate a response including a diuretic or intravenous fluid infusion. Diuretic or intravenous fluid infusion may be altered in response to continuously measured urinary output values. A processor may include an algorithm configured to alter a rate of diuretic or intravenous fluid infusion in response to measured urinary output values.

In some embodiments, electrophysical information may be monitored by a regulatory system. Electrophysical information, in the context of this application, refers to electrical phenomena associated with a physiological process or condition. Sensors positioned in a flexible conduit may measure electrophysical information. A processor may generate a response in reaction to measured electrophysical information. Responses may include delivery of pharmaceuticals and/or altering a machine function. A processor may alter an amount of chronotropic drugs or paralytic agents delivered to a body in response to measured electrophysical information. In certain embodiments, when measured electrophysical information is greater than a desired range, a processor may increase an amount of paralytic agent delivered to a body. Electrophysical properties of a body may decrease in response to an increased amount of delivered paralytic agent. An amount of paralytic agent, sedative, or anti-seizure delivered to a body may be continuously modified in response to continuously monitored electrophysical properties. In an embodiment, a next dose of paralytic agent, sedative, or anti-seizure delivered to a body may be modified in response to continuously monitored electrophysical properties. When electrophysical properties are measured again, the electrophysical information may be within a desired range.

In certain embodiments, a regulatory system may monitor blood gas levels. A flexible conduit may be positioned in a vascular structure of a body. Flexible conduit may include a pulse oximeter positioned along a length of the flexible conduit. In one embodiment, flexible conduit may be a triple lumen catheter, such as, but not limited to, a Swan Ganz catheter. One or more sensors in a flexible conduit may be configured to measure oxygen and/or carbon dioxide levels in the blood. A sensor in a flexible conduit may measure pH. A processor may continuously display blood oxygen and/or carbon dioxide levels. When oxygen levels are lower than a desired range, a processor may alter a ventilator function such that more oxygen is delivered to a body. In certain embodiments, a response from a processor may include pharmaceutical delivery in addition to altering a machine function. As oxygen and/or carbon dioxide levels in the blood increase and/or decrease, a processor may alter the ventilator function such that oxygen and/or carbon dioxide levels in the blood approach a desired range.

In some embodiments, a regulatory system may be configured to monitor and control cerebral infusion and/or intracranial pressure. When a measured intracranial pressure is greater than a desired range, a processor may deliver a pharmaceutical to a body that may decrease intracranial pressure. As intracranial pressure changes, a processor may modify rates of pharmaceutical infusion. A pharmaceutical configured to decrease intracranial pressure may include mannitol. A processor may change a ventilator function such that intracranial pressure decreases. In one embodiment, a processor may deliver a pharmaceutical so that blood pressure in a body increases. A processor may monitor blood pressure and intracranial pressure. A processor may generate one or more responses such that a measured blood pressure may be modified to optimize cerebral perfusion pressure. Cerebral perfusion pressure, in the context of this application, refers to mean arterial blood pressure minus intracranial pressure. Increasing blood pressure so that it is above intracranial pressure may decrease the likelihood of permanent brain injury due to high intracranial pressure.

Some pharmaceuticals may increase intracranial pressure. In an embodiment, a response generated by a processor may increase intracranial pressure by delivery of a pharmaceutical. It may be desirable to maintain a low intracranial pressure. A processor may generate a second response to control and/or maintain intracranial pressure in a desired range, such as decreasing an infusion rate of the delivered pharmaceutical that increased intracranial pressure, commencing infusion of mannitol or a similar pharmaceutical, and/or altering a ventilator function.

In some embodiments, a regulatory system may be configured to monitor and control cardiac output. A flexible conduit, such as a Swan catheter, of a regulatory system may float in a vein. A flexible conduit such an arterial catheter may also be used to measure cardiac output. One or more sensors positioned in flexible conduit may be configured to measure cardiac output. A sensor may be a flow meter. A processor may generate one or more responses including, but not limited to, infusing pharmaceuticals in a body to modify cardiac output to be in a desired range.

A regulatory system may measure and control hemodynamic parameters including blood pressure. A flexible conduit may be an arterial line and/or a cardiac output monitor. A regulatory system may include an intracranial pressure monitor. Sensors in an arterial line may continuously measure arterial pressure. Infusion of pharmaceuticals configured to modify blood pressure may be controlled as a function of trends in arterial pressure. A processor may include an algorithm to maintain blood pressure in a desired range such as 65-70. A processor may include an algorith to maintain a cerebral perfusion pressure greater than 60. Pharmaceuticals may be continuously or intermittently infused in a patient in response to changes in blood pressure.

In an embodiment, a regulatory system may at least monitor blood serum levels of phenytoin. A regulatory system may include a central venous catheter positioned in a body. A regulatory system may also include an electroencephalogram (EEG). Continuous EEG measurements may be sent to processor. A processor may monitor EEG measurements for signs of seizure activity. A processor may determine the optimum serum phenytoin dose. Through continuous measuring and monitoring of serum phenytoin levels, variations in serum phenytoin levels may be substantially minimized and a risk of seizure may be decreased.

In some embodiments, a regulatory system may be configured to monitor and control induced comas. Sedatives, paralytic agents, and/or phenobarbitals may induce comas. Phenobarbital and/or a paralytic agent may be continuously delivered from a pharmaceutical storage unit. Pharmaceutical storage unit may be coupled to a processor. One or more sensors of a regulatory system may measure one or more physiological responses to an electric signal. Regulatory system may include sensors that measure other physiological conditions. In one embodiment, a processor may be coupled to an EEG. An EEG may detect and record patterns of electrical activity. A processor may check for abnormal electrical activity. A processor may analyze measured physiological responses and generate one or more responses such as modifying a rate of phenobarbital and/or paralytic agent infusion. If a measured physiological response drops below a critical level, then a processor may generate one or more responses including decreasing an infusion rate of phenobarbital and/or paralytic agent, commencing infusion rate of a pharmaceutical, and/or altering one or more machine functions. A critical level may be a level determined by the FDA as dangerous for a human. A critical level may be input by a user into a processor. By continuously monitoring electrical activity, phenobarbital and/or paralytic agent delivery rates may be monitored and controlled by a processor such that a patient substantially maintains a phenobarbital coma and/or overdosing on phenobarbitals and/or paralytic agent is substantially inhibited. Preventing overdose may include substantially reducing the probability of a patient requiring more than 4 days to wake up from an induced coma and/or substantially reducing the probability of death from high levels of phenobarbitals and/or paralytic agents in a body.

A processor may include a local computer system, including, but not limited to, a personal desktop computer. A processor may include remote systems or may include computers connected over a network. In some embodiments, a wide area network (“WAN”) and/or local area networks (“LANs”) may couple a plurality of regulatory systems. WAN typically includes a plurality of computer systems that may be interconnected through one or more networks. WAN may include a variety of heterogeneous computer systems and networks that may be interconnected in a variety of ways and that may run a variety of software applications. LAN may be a network that spans a relatively small area. Each node (i.e., individual computer system or device) on LAN may have its own CPU with which it may execute programs, and each node may also be able to access data and devices anywhere on LAN. LAN, thus, may allow many users to share devices (e.g., printers) and data stored on file servers. LAN may be characterized by a variety of types of topology (i.e., the geometric arrangement of devices on the network), of protocols (i.e., the rules and encoding specifications for sending data and whether the network uses a peer-to-peer or client/server architecture), and of media (e.g., twisted-pair wire, coaxial cables, fiber optic cables, and/or radio waves). Data obtained during monitoring of an individual patient or from a collection of data from previous and/or simultaneous patients may be used to drive the algorithms as the computer system learns to anticipate the physiologic response to alterations in therapy.

Several regulatory systems may transmit measured physiological condition data to a centralized processor. A centralizer processor may be one or more personal computers or mainframe computer systems coupled to a LAN and/or WAN. A centralizer processor may be coupled to a storage device or file server and mainframe terminals. Mainframe terminals may access data stored in the storage device or file server coupled to or included in mainframe computer system. A processor or central processor may be operable to execute the computer programs to implement method for implementing one or more regulatory systems for measuring, monitoring, and controlling physiological conditions.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description to the invention. Changes may be made in the elements described herein without departing from the spirit and scope of the invention as described in the following claims. In addition, it is to be understood that features described herein independently may, in certain embodiments, be combined.

What is claimed is:

1. A regulatory system for monitoring and altering physiological conditions of a human body comprising:
a flexible conduit, wherein a portion of the flexible conduit is positionable in a human body;
one or more sensors positionable in the flexible conduit, wherein at least one sensor is configured to measure one or more physiological conditions of the human body; and
a processor coupled to at least one of the sensors, wherein the processor is configured to generate one or more responses, wherein the one or more responses maintain a physiological condition of the human body within a desired range.

2. The system of claim 1, wherein at least a portion of the flexible conduit is positionable in a vasculature of the human body.

3. The system of claim 1, wherein at least a portion of the flexible conduit is positionable in a cavity of the human body.

4. The system of claim 1, wherein at least one response comprises delivering one or more pharmaceuticals to the human body.

5. The system of claim 2, wherein at least one of the pharmaceuticals is selected to alter at least one of the physiological conditions such that at least one of the measured physiological conditions is within a desired range.

6. The system of claim 1, further comprising a pharmaceutical storage unit, wherein the pharmaceutical storage unit comprises one or more pharmaceuticals, and wherein at least one response comprises delivering one or more pharmaceuticals from the storage unit to the patient.

7. The system of claim 1, wherein the desired range of at least one of the physiological conditions is determined by FDA guidelines.

8. The system of claim 1, further comprising supplying the desired range of at least one measured physiological condition to the processor.

9. The system of claim 1, wherein at least one of the physiological conditions comprises blood glucose level, and wherein at least one response comprises delivering insulin to the patient.

10. The system of claim 1, wherein at least one of the physiological conditions comprises blood pressure, and wherein at least one response comprises delivering a pharmaceutical configured to modify the blood pressure of a human body such that the blood pressure of the human body is maintained substantially in a desired range.

11. The system of claim 1, wherein at least one response comprises controlling one or more machines, wherein at least one of the machines is configured to alter at least one of the physiological conditions of the human body.

12. The system of claim 1, wherein at least one response comprises modifying delivery of oxygen from a ventilator to the human body.

13. The system of claim 1, wherein the one or more physiological conditions comprise a plurality of physiological conditions.

14. The system of claim 1, further comprising a display, wherein at least one of the measured physiological conditions is indicated on the display.

15. The system of claim 1, further comprising one or more indicators, wherein the processor is configured to activate at least one of the indicators when at least one of the measured physiological conditions deviates from a desired range.

16. The system of claim 1, wherein the flexible conduit comprises an intravenous catheter.

17. The system of claim 1, wherein the flexible conduit comprises a central line.

18. A method of monitoring one or more physiological conditions of a human body comprising:
measuring one or more physiological conditions of a human body with one or more sensors;
monitoring one or more physiological conditions with a processor;
automatically controlling at least one of the measured physiological conditions such that at least one of the measured physiological conditions is maintained substantially in a desired range.

19. The method of claim 18, further comprising analyzing a measured physiological condition to determine whether at least one of the measured physiological conditions deviates from a desired range.

20. The method of claim 18, wherein controlling at least one of the measured physiological conditions comprises generating one or more responses, and wherein a processor coupled to at least one sensor generates the one or more responses.

21. The method of claim 18, wherein controlling at least one of the measured physiological conditions comprises automatically delivering one or more pharmaceuticals to the human body.

22. The method of claim 18, wherein controlling at least one of the measured physiological conditions comprises adjusting a machine, wherein the machine is configured to modify at least one of the measured physiological conditions of the human body.

23. The method of claim 18, wherein measuring one or more measured physiological conditions of the human body comprises measuring a plurality of measured physiological conditions substantially simultaneously.

24. The method of claim 18, wherein controlling at least one of the measured physiological conditions comprises controlling a plurality of measured physiological conditions substantially simultaneously.
